(19) United States
(12) Patent Application Publication
(10) Pub. No.: US 2009/0112245 A1
(43) Pub. Date: Apr. 30, 2009

(54) Device and method for the performance of ophthalmological operations

(76) Inventor: Eduard Anton Haefliger, Basel (CH)

Correspondence Address: GREER, BURNS & CRAIN, 300 S WACKER DR, 25TH FLOOR, CHICAGO, IL, 60606, US

(21) Appl. No.: 12/346,326

(22) Filed: Dec. 30, 2008

Related U.S. Application Data
(62) Division of application No. 10/850,722, filed on May 21, 2004.

(30) Foreign Application Priority Data
Nov. 22, 2001 (IB) PCT/IB01/02224

(51) Int. Cl. A61F 9/007

(52) U.S. Cl. 606/170

(57) ABSTRACT

The invention relates to a device for performing ophtalmological operations, especially for treating glaucoma. Said device comprises a probe in which a suction channel is arranged. A cutting tool is arranged in the suction channel, by which means separating tissue extending to Schlemm's canal and being sucked into the suction channel can be removed. The device enables openings to be created between the front chamber and Schlemm's canal in a targeted and careful manner.

### PRIORITY CLAIM

This Application is a divisional of and claims the benefit if U.S. patent application Ser. No. 10/850,722 filed on May 21, 2004.

### CROSS REFERENCES TO RELATED APPLICATIONS

This application claims the priority of international patent application PCT/IB01/02224, filed Nov. 22, 2001, the disclosure of which is incorporated herein by reference in its entirety.

### TECHNICAL FIELD

The invention relates to a device and a method for treating glaucoma by generating openings between the anterior aqueous chamber and Schlemm's canal according to the preamble of the independent claims.

WO 01/37767 describes several devices for treating of glaucoma. These devices are provided with a probe which can be introduced into the anterior aqueous chamber of the eye in order to generate openings in the tissue separating the anterior aqueous chamber and Schlemm's canal. In an embodiment this probe has a channel which e.g. can be connected to a suction unit or an irrigation unit. In addition to this channel, a cutting tool, a gripping tool or a clamping tool can be present in order to create openings in the separating tissue.

Such a device can not guarantee that only the separating tissue between the anterior aqueous chamber and Schlemm's canal is destroyed. Even the most careful handling can e.g. lead to the destruction of tissue which is behind Schlemm's canal i.e. tissue located on the side of Schlemm's canal facing away from the anterior aqueous side. Furthermore, tissue which has been cut off can get into Schlemm's canal.

### DISCLOSURE OF THE INVENTION

Hence, it is a task of the invention to provide a device or a method, respectively, which allow a reliable creation of openings in the separating tissue to Schlemm's canal.

This task is solved by the independent claims.

The invention is based on the thought that a suction channel in the probe can be used to aspirate the separating tissue to be removed and then to remove the tissue which is aspirated. In a first aspect of the invention this is achieved by arranging the cutting means directly in the suction channel allowing a targeted detachment in the area of the suction channel. In a second aspect, the separation means is adapted for detaching the separating tissue along a line which essentially extends along a longitudinal axis of the suction channel. This allows as well a targeted detachment in the area of the suction channel. In this manner it is possible to suck off the tissue which has been removed through the suction channel. Furthermore, the suction effect supports the separation process.

Preferably the separation means is formed as cutting tool which allows a mechanical transection of the separating tissue.

In a preferred embodiment the separation edge or the cutting edge, respectively, of the separation means is arranged in a stationary manner in the suction channel or its orifice. It is positioned so that the separating tissue to be cut off can be drawn to the cutting edge by low pressure in the suction channel. In this embodiment a mechanical movement of the separation means is possibly not necessary.

Preferably the suction channel is provided with a cutting tool having a cutting edge extending maximally to the orifice of the suction channel. This has the advantage that the cutting edge exerts its effect only on tissue which is sucked into the suction channel. Thus, accidental tissue injury can be avoided. In particular, tissue located behind Schlemm's canal is not injured.

### BRIEF DESCRIPTION OF THE DRAWINGS

Further embodiments, advantages and applications of the invention result from the dependent claims and the now following description referring to the figures. Herein show:

FIG. 1 a graphic representation of the device according to the invention in a treatment of glaucoma by means of a sectional figure through the anterior aqueous chamber of an eye,

FIG. 2 a longitudinal section of a first embodiment of the invention,

FIG. 3 the embodiment of FIG. 2 with a cutting tool in the separation position,

FIG. 4 a longitudinal section of a second embodiment of the invention,

FIG. 5 a longitudinal section of a third embodiment of the invention,

FIG. 6 the embodiment of FIG. 5 with a cutting tool in separation position,

FIG. 7 a longitudinal section of a fourth embodiment of the invention,

FIG. 8 a longitudinal section of a fifth embodiment of the invention,

FIG. 9 a longitudinal section of a sixth embodiment of the invention and

FIG. 10 a longitudinal section through the proximal part of an embodiment of the invention with a piezoelectric power unit.

### MODES FOR CARRYING OUT THE INVENTION

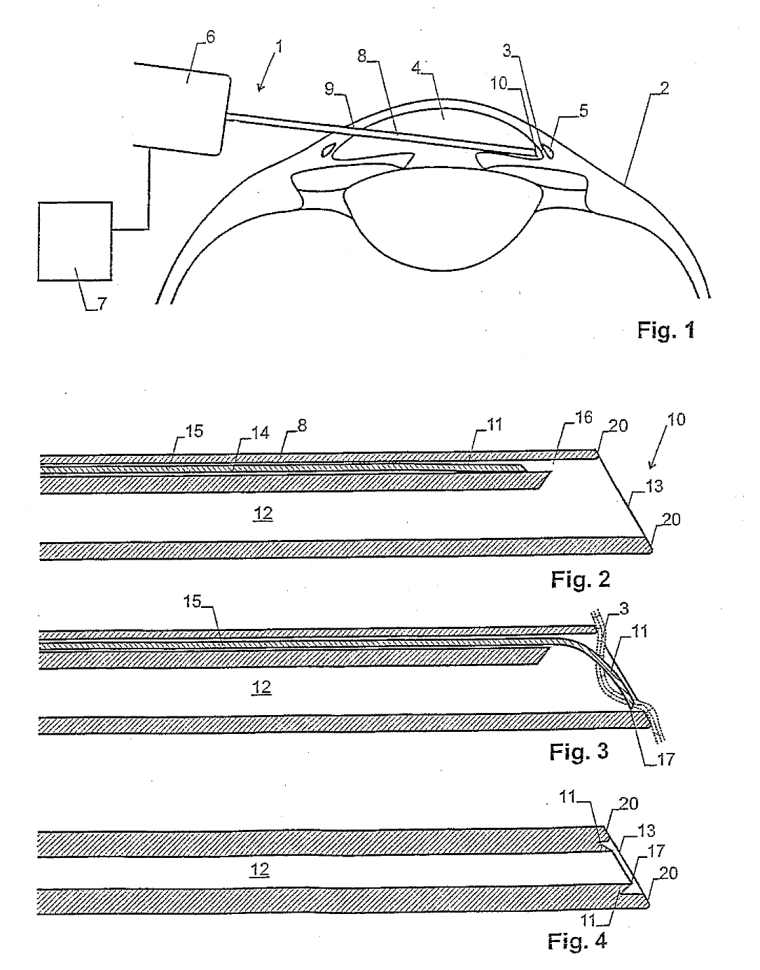

In FIG. 1 a device 1 for performing microsurgical operations in an eye 2 is shown. The device is particularly suitable to create additional openings in the separating tissue 3 between the anterior aqueous chamber 4 and Schlemm's canal 5. Such openings improve the efflux of liquids from the anterior aqueous chamber 4 and are made to reduce high-pressure in the eye 1 and to treat glaucoma.

The device 1 comprises a handle 6, a schematically depicted suction device 7 and a probe 8. The probe 8 possesses a length, width and shape which e.g. allow that the probe can be inserted through a prepared slide 9 in the anterior aqueous chamber 4, and in a manner so that the distal end 10 can be guided to the separating tissue 3 between the anterior aqueous chamber 4 and Schlemm's canal 5.

The probe 8 possesses essentially the shape of a needle. It can be straight or bended.

FIGS. 2 and 3 show a longitudinal section through an embodiment of the probe 8 in the area of the end 10. As can be seen, the probe 8 has a suction channel 12 which extends from the orifice 13 at the end 10 through the whole probe 8 to the handle 6 and is connected to the suction device 7. A low-pressure can be generated in the suction channel 12 by switching on the suction device.

As can be seen from the figures, the orifice 13 is arranged frontally at the distal end of probe 8. This allows to guide the orifice in an easy manner to the separation wall of Schlemm's canal. As can be seen from the figures as well, the orifice 13 is preferably not perpendicular but inclined to the longitudinal axis of the probe. This measure as well simplifies the guidance of the orifice to the separation wall of Schlemm' s canal as can be seen in particular from FIG. 1.

In the device depicted in FIG. 2 and FIG. 3 a further channel 14 extends along the probe 8, in which a longitudinally displaceable separation device or cutting device 15, respectively, is arranged. FIG. 2 shows the separation device or the cutting device 15, respectively, in neutral position, FIG. 3 in separation position. In order to displace the separation device or the cutting device 15, respectively, from the neutral position to the separation position an appropriate operating means in handle 6 can be provided (not shown).

The distal end of the separation device or the cutting device, respectively, forming the actual separation means or the cutting tool 11, is bended towards the suction channel 12. When the separation device or the cutting device 15, respectively, is moved from neutral position to separation position, the cutting tool 11 appears through a provided opening 16 lying between the suction channel 12 and the additional channel 14 in the suction channel 12 in an area of the orifice 13. Thereby it moves over the crossection of the suction channel 12, and cuts the separating tissue 3 sucked into the orifice by means of a cutting edge 17 at the end 15a and creates the desired opening. The part of the separating tissue 3 which has been cut off can be sucked off through the suction channel 12.

A second embodiment of the device according to the invention is depicted in FIG. 4. In this embodiment the separation means or the cutting tool 11, respectively, are formed by a circular or oval cutting edge 17, respectively, which extends along the inner wall of suction channel 12. The cutting edge 17 is arranged closely to the orifice 13 or at the orifice 13. When separating tissue 3 is sucked into the orifice 13 it meets the cutting edge 17 and is cut off.

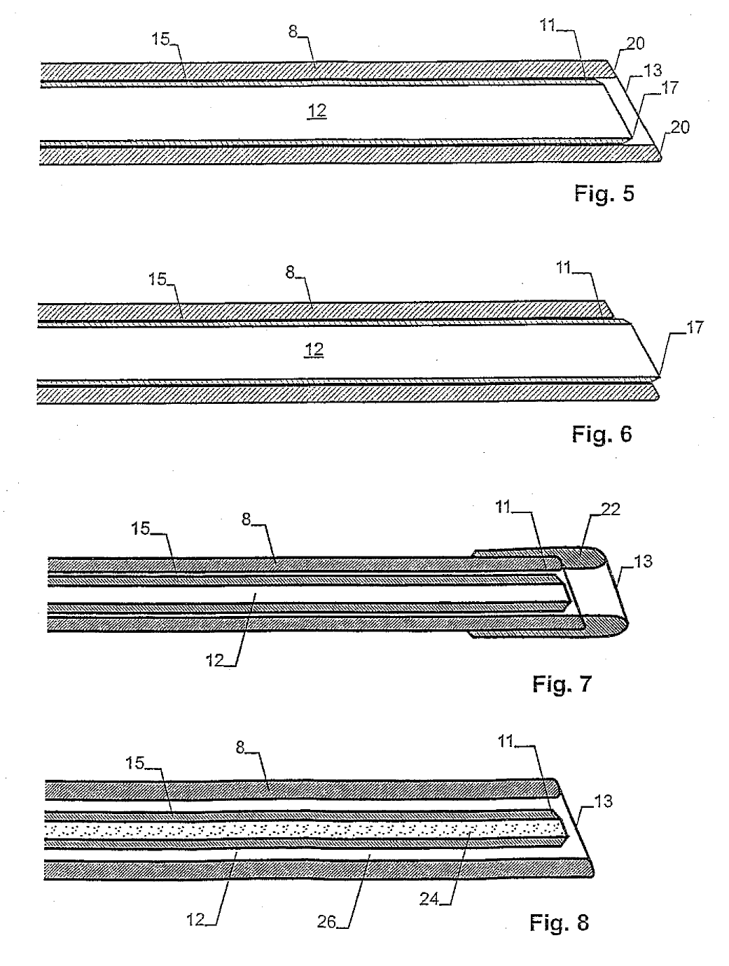

A third embodiment of the device according to the invention is depicted in FIGS. 5 and 6. In this embodiment the cutting tool 15 is formed by a tube section which is arranged in the suction channel 12. The distal end of the tube forms the cutting tool 11 with cutting edge 17.

Preferably the cutting device 15 or the cutting tool 11 is longitudinally displaceable and can be moved from the neutral position shown in FIG. 5 to the separation position shown in FIG. 6. In the neutral position the cutting edge 17 is behind the orifice 13, in the separation position the cutting edge 17 protrudes beyond the orifice 13 up to a given distance. The distance corresponds approximately to the typical width of the separating tissue 3. In this manner, separating tissue which is sucked in by the orifice 13 can be cut off.

It is conceivable that the cutting device 15 is rotatable about its longitudinal axis so that it can be turned at least over a limited angular range compared to the probe 8. This allows to achieve an additional cutting effect by turning back and forth the cutting device.

In the second and third embodiment shown herein, the cutting edge 17 extends around the circumference of the suction channel 12 along its inner side. It is as well conceivable that the cutting edge 17 is divided into several parts which rotate about the longitudinal axis of suction channel 12 and does not form a continuous edge. Preferably, the cutting tool cuts off the separating tissue 3 along an essentially circular cutting line.

As can be seen from FIGS. 2 to 6, the edges of orifice 13 are preferably blunt or blunted, in contrary to the cutting edge 17 which preferably tapers in an acute angle. Furthermore, the orifice 13 is surrounded by an essentially flat shoulder surface 20 on which the separating tissue can rely on. Thereby it is achieved that the separating tissue 3 is cut off by the effect of the cutting edge 17. It is as well possible to arrange the cutting edge 17 such that it forms the lateral edge of orifice 13.

The suction channel 12 can have a round or square crossection. In particular in the first embodiment according to FIG. 2 and FIG. 3 the crossection is at least in the area of the distal end 10 rather square so that the cutting tool can glide over the complete crossection while in the embodiments according to FIGS. 4 to 6 the crossection is preferably rather round or elliptic.

In a further embodiment as shown in FIG. 7, a sleeve 22 is arranged as support device at the distal end of the probe 8. It is made of a material which is more deformable than the probe and allows a more tight seal of the suction channel 12 towards the tissue.

In the application of the device described herein, the distal end 10 of probe 8 is guided through the anterior aqueous chamber 4 to separating tissue 3 whereby the cutting tool 11 of the embodiments according to FIG. 2 and FIG. 3 or FIGS. 5 and 6, respectively, is at this stage in neutral position. Then, a piece of separating tissue is sucked into the orifice 13 of the suction channel 12. In the embodiment according to FIG. 4 it is cut off automatically by the cutting edge 17. In the embodiment according to FIGS. 2 and 3 or 5 and 6, respectively, the cutting device 15 is moved to the separation position so that the separating tissue which is sucked into is cut off. When the separation is incomplete, then the remaining piece of tissue can be removed by appropriate forceps.

While the probe 8 is guided through the anterior aqueous chamber 4 of the eye to the separating tissue 3, the suction device operates at a relatively low capacity or is switched off. As soon as the probe is in contact with separating tissue 3, a preferably short “suction pulse” having a higher suction capacity is generated during which the separating tissue can be detached. When the suction device 7 is operated at a low capacity during the insertion, it is possible to determine based on the pressure drop and/or the reduction of the suction rate when the probe 8 is in suction contact with the separating tissue 3.

Therefore, the suction device is preferably constructed such that it is able to generate short suction pulses of e.g. 50 to 200 ms. For an operation at low capacity during the insertion, it has to be constructed such that it allows the generation of short suction pulses of a first higher suction capacity and the generation of a permanent suction at a second lower suction capacity. The construction of such a pump with an appropriate pump control and corresponding controls to activate the suction pulse are within the skills of a person skilled in the art.

When the separation or the cutting tool 11, respectively, more precisely the cutting edge 17, is arranged in the suction channel 12 as shown in the embodiments according to FIG. 2 to 4, and it extends maximally to orifice 13, then only tissue which is sucked into the suction channel 12 can be cut off. Thus, an accidental injury of tissue e.g. at the outside of Schlemm's canal 5, can be avoided. In the embodiment of FIG. 6 an accidental injury is avoided in that the cutting edge 17 can only be displaced beyond orifice 13 up to a distance comparable to the width of the separating tissue 3.

In the devices shown so far, the separation means 11 is formed by a cutting tool which cuts off the separating tissue by a purely mechanical cut. However, it is possible to cut off the separating tissue which has been sucked in, in a different manner or to support the cutting procedure by other means.

FIG. 8 shows an embodiment in which the separation or the cutting device 15, respectively, is again formed by a tube in the probe 8 wherein in this embodiment the inside of the separation or cutting device 15 contains an etching agent 24 (e.g. an acid or a base). In this embodiment, the suction channel 12 is located between the separation or cutting device 15, respectively, and the wall of the probe 8.

In this embodiment a piece of separating tissue is sucked into the orifice 13 as well. Then, a small amount of the etching agent 24 is pumped by a micropump in handle 6 towards the distal end where it gets in contact with the separating tissue and at least partially dissolves or cuts off the separating tissue. The etching agent is sucked off via the suction channel 12 before it can leak into other parts of the eye.

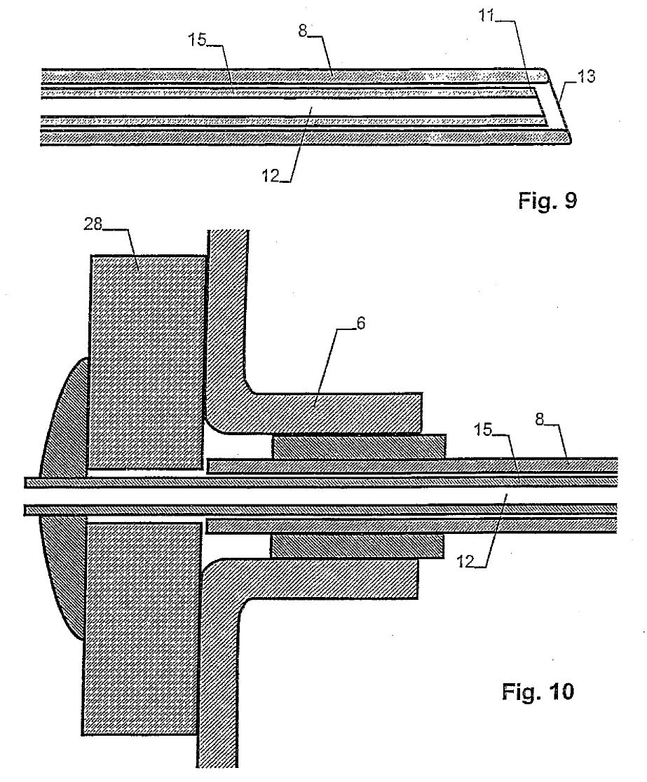

In the embodiment according to FIG. 9 the separation or cutting device 15, respectively, is formed by a tubular optical waveguide which contains in its middle the suction channel 12. Separating tissue is sucked into orifice 13 as well. Then, an optical light pulse of high intensity is sent through the waveguide leaving the waveguide at its distal end and where it cuts off the separating tissue.

Instead of a tubular optical waveguide a normal waveguide in form of a fiber can be used which e.g. runs in the suction channel 12 formed by the probe.

The optical, chemical and mechanical separation methods shown herein can be combined as well.

In all cases it can be reasonable to make the separation means 11 and/or the probe vibrate, in particular by frequencies of the supersonic range. The vibration of the separation means 11 supports the separation procedure. The vibration of the separation means 11 and/or of the probe 8 can avoid that the suction channel 12 is blocked by tissue which has been sucked in.

FIG. 10 shows an embodiment of the device in form of a section through the proximal end of probe 8, in which the separation device or the cutting device 15, respectively, can be oscillated. To this end a piezoelectric transducer is provided which holds on one side the proximal end of the separation or cutting device 15, respectively, and on the other side e.g. is attached to the housing of the handle 6. The shaft 8 can as well rely on the handle 6. The separation or cutting device 15 can be oscillated by supplying an alternating voltage to the transducer 28.

In the embodiments shown herein, the diameter of orifice 13 is preferably between 50 and 200 μm. Since Schlemm's canal is generally not larger than 200 μm, larger diameters are unfavorable.

For the construction of the device described herein conventional mechanical processing steps can be used. However, in particular the distal end of the separation means 11 can be produced using anisotropic etched methods e.g. it can be formed of silicon.

While there are shown and described presently preferred embodiments of the invention, it is to be distinctly understood that the invention is not limited thereto but may be otherwise variously embodied and practiced within the scope of the following claims.

1. A device for treating glaucoma by creating openings between the anterior aqueous chamber and Schlemm's canal of an eye, comprising:
a probe adapted for insertion in the anterior aqueous chamber such that one end of the probe is guidable to a separating tissue between the anterior aqueous chamber and Schlemm's canal;
a separation means arranged at the end of the probe for cutting the separating tissue;
a suction device for the generation of suction pulses for the aspiration of separating tissue; and
a suction channel in communication with said suction device and extending through the probe and ending in an orifice at the end of the probe,
wherein the separation means is arranged in the suction channel at the orifice such that the separating tissue sucked into the suction channel is removable by the separation means.

2. The device of claim 1, wherein the suction pulses last maximally 200 ms.

3. The device of claim 1, wherein the suction pulses are between 50 ms and 200 ms.

4. The device of claim 1, wherein the suction device is adapted for the generation of suction pulses of a first higher suction capacity and a permanent suction at a second lower suction capacity.
